(19) United States
(12) Patent Application Publication
(10) Pub. No.: US 2020/0138838 A1
(43) Pub. Date: May 7, 2020

(54) COMPOSITION COMPRISING HMSS/HMOS AND USE THEREOF

(71) Applicant: Glycom A/S, Hørsholm (DK)

(72) Inventors: Bruce McConnell, La Tour de Peilz (CH); Louise Kristine Vigsnæs, København NV (DK); Gabriela Bergonzelli, Bussigny (CH); Laurent Favre, Carrouge (CH); Laurent Ferrier, Toulouse (CH); Clara Lucia Garcia-Rodenas, Forel (CH)

(21) Appl. No.: 16/706,627

(22) Filed: Dec. 6, 2019

Related U.S. Application Data
(63) Continuation-in-part of application No. 15/906,911, filed on Feb. 27, 2018.
Application No. 15/906,911 is a continuation-in-part of application No. 15/147,115, filed on May 5, 2016, now abandoned.
Application No. 15/147,115 is a continuation-in-part of application No. 15/034,593, filed on May 5, 2016, now abandoned.

(30) Foreign Application Priority Data
Oct. 29, 2014 (DK) PA 2014 70663

(51) Int. Cl. A61K 31/702; A61P 29/02; A61P 1/00

(52) U.S. Cl. CPC A61K 31/702; A61P 1/00; A61P 29/02

(57) ABSTRACT

A method, in one embodiment, includes administering to a non-infant human an effective amount of a combination of the human milk oligosaccharides (“HMO”s) 2′-FL and DFL, where the amount of the combination of HMOs is effective for modulating the microbiota of the non-infant human and reducing one or more post-treatment condition such as pain and/or a perception of visceral pain in the non-infant human.

### CROSS-REFERENCES TO RELATED APPLICATIONS

This is a continuation-in-part application of and claims priority to U.S. patent application Ser. No. 15/906,911 entitled “Composition Comprising HMSs/HMOs and use thereof” and filed on Feb. 27, 2019 for Louise Kristine Vigsnæs et al, which is a continuation-in-part application of and claims priority to U.S. patent application Ser. No. 15/147,115 entitled “Composition Comprising HMSs/HMOs and use thereof” and filed on May 5, 2016 for Louise Kristine Vigsnæs et al, which is a continuation application of and claims priority to U.S. patent application Ser. No. 15/034,593 entitled “Synthetic composition and method for treating irritable bowel syndrome”, filed on May 5, 2016 for Thierry Hennett, which is a national stage filing in accordance with 35 U.S.C. § 371 of PCT/DK2015/050332 filed Oct. 29, 2015, which claims the benefit of the priority of Denmark Patent Application No. PA 2014 70663, filed Oct. 29, 2014, all of which are incorporated herein by reference.

### FIELD

This invention relates generally to compositions and methods for managing abdominal pain including perception of pain.

### BACKGROUND

Visceral hypersensitivity including abdominal pain is a major clinical problem but far less is known about is mechanisms than somatic pain. Visceral hypersensitivity is defined as pain felt arising from the internal organs (viscera) of the body, including the intestines. There are multiple aetiologies for pain sensed in viscera, including inflammation (acute and chronic), infection, disruption of normal mechanical processes (e.g. gastrointestinal dysmotility), alterations in nerves and dysfunctions in gut-brain functioning. The recent growth in interest in pain originating from viscera reflects an important paradigm shift in the awareness of the magnitude and impact of visceral pain disorders.

Visceral pain is associated with many disorders. For example, abdominal pain is a defining criterion for irritable bowel syndrome (IBS). IBS is a clinically heterogeneous disorder of human patients, particularly adult, with chronic symptoms such as abdominal pain, abdominal discomfort, abdominal bloating, fatigue, and changes in bowel movement patterns, such as patterns of loose or more frequent bowel movements, diarrhea and constipation. Routine clinical tests on patients typically show no abnormalities, although their bowels may be more sensitive to certain stimuli, such as balloon insufflation testing. The worldwide prevalence of IBS is about 10-20% but may be higher in certain countries (Longstreth et al. Gastroenterology 130, 1480 (2006)). The causes of IBS are unknown but disruptions of the brain-gut axis, acute gastrointestinal infections, small intestinal bacterial overgrowths, antibiotic usages, and dysbiosis are thought to be important risk factors (Kim et al. Dig. Dis. Sci. 57, 3213 (2012)). Other risk factors are young age, prolonged fever, anxiety, and depression. Chronic low-grade inflammation commonly occurs in IBS patients, but there are otherwise little or no observable clinical manifestations. Abdominal pain is also associated with many other functional gastrointestinal disorders such as non-coeliac wheat sensitivity, abdominal migraine, recurrent abdominal pain, and, in infants, colic.

The diagnosis of functional gastrointestinal disorders is difficult. There are no biomarker-based tests for diagnosis. Instead, diagnosis generally involves excluding conditions that produce similar symptoms and then following a procedure to categorize a patient's symptoms. For example, for IBS, ruling out parasitic infections, lactose intolerance, and coeliac disease is recommended for all patients before a diagnosis is made. Once diagnosed, IBS patients are usually classified in accordance with the Rome IV criteria into four symptom subtypes based on stool consistency: diarrhea-predominant (IBS-D), constipation-predominant (IBS-C), mixed subtype (IBS-M or IBS-A) with alternating episodes of both diarrhea and constipation, and unsubtyped IBS (IBS-U). The diagnosis of other functional gastrointestinal disorders generally follows similar procedures.

There is no cure for functional gastrointestinal disorders and current treatments focus on attempting to relieve symptoms; including visceral pain but current pain treatments have limited efficacy. Treatments take various forms such as dietary adjustments, medication, and psychological interventions. Patient education and good doctor-patient relationships are also important. However, most treatment is unsatisfactory and most patients continue to experience chronic pain, fatigue, and other symptoms. While functional gastrointestinal disorders have no direct effect on life expectancy, their high prevalence and significant effects on quality of life make them conditions with a high social cost. In particular, the general hopelessness associated with IBS is a source of frustration for both patients and health care practitioners treating them.

Visceral pain usually has a temporal evolution and clinical features vary in different phases of pathology. ‘True visceral pain’ arises as a diffuse and poorly defined sensation usually perceived in the midline of the body, at the lower sternum or upper abdomen. In patients, pain from different visceral organs can have different areas of presentation, e.g. bladder to perineal area, heart to left arm and neck, left ureter to left lower quadrant and loin. This diffuse nature and difficulty in locating visceral pain are due to a low density of visceral sensory innervation and extensive divergence of visceral input within the central nervous system. Visceral pain is therefore perceived more diffusely than noxious cutaneous stimulation with respect to location and timing. Visceral pain is often associated with marked autonomic phenomena, including pallor, profuse sweating, nausea, gastrointestinal disturbances and changes in body temperature, blood pressure and heart rate (Sikandar et al. Curr. Opin. Support. Palliat. Care 6, 17 (2012)).

The “brain-gut axis” is a theoretical model depicting bidirectional neural pathways linking cognitive, emotional and autonomic centers in the brain to neuroendocrine centers, the enteric nervous system, and the immune system. The gastrointestinal tract possesses both the largest neural network outside the brain and the most extensive immune system, which brings about the opportunity for crosstalk between neurons and immune cells, including mast cells. One potential mechanism for visceral pain involves mast cells. Many studies have confirmed that endings of postganglionic sympathetic, peptidergic and vagal fibres, and enteric neurons, are in close proximity to mast cells. For example, it has been estimated that 70% of intestinal mucosal mast cells are in direct contact with nerves. Due to their location mast cells play an important role in the regulation of gastrointestinal visceral hypersensitivity and vascular permeability, and an alteration in the mast cells—nerve axis can contribute to autonomic dysregulation of the gut and associated pain and perceptual changes in visceral disorders. Several studies have noted an increased number of mast cells in the mucosa of patients with gastrointestinal diseases such as irritable bowel syndrome, mastocytic enterocolitis, and systemic mastocytosis (Buhner et al. Biochim. Biophys. Acta 1822, 85 (2012)).

The functional mast cell-neuronal units consist of 2 pathways: the nerve to mast cells signaling and the mast cells to nerve signaling. One of the important mediators in this process is mast cell tryptase, which activates PAR2 present on sensory afferents. PAR2 plays a crucial role in sensitizing afferent neurons and causes the release of substance P and CGRP. This system acts to amplify the inflammatory response with the gastrointestinal tract and leads to increased motility and secretion as part of the ENS response to mast cell degranulation. Mast cells hyperplasia and activation can lead to abnormal gastrointestinal sensitivity, motility, and secretion, which in turn contribute to the hallmark symptoms found in functional gastrointestinal disorders including abdominal pain and/or discomfort, bloating, and abnormal bowel function (diarrhea and/or constipation) (Zhang et al. J. Neurogastroenterol. Motil. 22, 181 (2016)).

Another potential mechanism for visceral pain involves the interaction between the intestinal microbiota and pathways mediating visceral pain. The absence of gastrointestinal bacteria, such as which occurs in germ-free mice, is associated with reduced perception of pain following different inflammatory stimuli. Further, modulation of the intestinal microbiota by the administration of various probiotics or prebiotics also has been shown to alter pain responses. The human gastrointestinal microbiota includes at least 1,000 species of bacteria, and about 1014 individual bacterial cells from about 160 different species inhabit each individual's intestine (Qin et al. Nature 464, 59 (2010)). It is believed that an individual's genetic make-up and acquired immunity, as well as environmental factors, influence their gastrointestinal microbiota. The microbiota, in turn, shape the individual's immunity and physiology within the gastrointestinal system. It is also believed that a healthy individual maintains a symbiotic relationship with the microbiota colonizing his/her intestines, while many individuals with functional gastrointestinal disorders have an imbalance in this microbiota-host interaction.

However, many gastrointestinal diseases are also associated with abdominal pain; not only functional gastrointestinal disorders. For example, diseases such as Crohn's Disease, ulcerative colitis, and infectious diarrhea.

Thus, the inventors have determined methods of managing pain and/or the perception of pain that safe and effective interventions that may be desirably used. Many pain management medications are associated with side effects which become problematic with chronic use. The inventors of the disclosure have determined, therefore, a particular interest to identify means of reducing nociception that are non-pharmacological and associated with low risk for the patient.

The use of human milk oligosaccharides in the management of IBS-associated pain has been proposed in WO2016/066175. Further, the use of human milk oligosaccharides in the management of visceral pain associated with mast cell activation has been proposed in WO2017/190755. Both publications disclose the use of one or more human milk oligosaccharides selected from a large variety of human milk oligosaccharides in the management of pain. The preferred human milk oligosaccharides are 2′-FL, 3-FL, DFL, LNnT, 3′-SL, 6′-SL or LNFP-I and the most preferred ones being a mix of 2′-FL and/or DFL, and LNnT or LNT.

However, the inventors have determined that there remains a need for safe interventions permitting the effective management of visceral pain, especially on a chronic basis.

### SUMMARY

This invention provides a synthetic composition comprising one or more human milk oligosaccharides (HMOs), that can be advantageously used in the management of visceral hypersensitivity and/or pain in a non-infant human.

Accordingly, in a first aspect, this invention provides a combination of the human milk oligosaccharides 2′-FL (2′-O-fucosyllactose) and DFL (difucosyllactose, 2′,3-di-O-fucosyllactose) for use in reducing pain and/or the perception of visceral pain in a non-infant human.

In a second aspect, this invention provides a synthetic composition consisting essentially of a combination of the human milk oligosaccharides 2′-FL and DFL for use in reducing pain and/or the perception of visceral pain in a non-infant human.

Preferably, the synthetic composition contains an amount of 1 g to 15 g of 2′-FL and DFL; more preferably 2 g to 10 g. For example, the synthetic composition may contain 3 g to 7 g of 2′-FL and DFL.

The synthetic composition may also include a bifidobacteria; for example, Bifidobacterium longum and/or Bifidobacterium bifidum.

In a third aspect, this invention provides a pack for use in in reducing pain and/or the perception of visceral pain in a non-infant human, the pack comprising at least 14 individual daily doses of an effective amount of a synthetic composition consisting essentially of a combination of the human milk oligosaccharides 2′-FL and DFL.

Preferably each daily dose in the pack contains an amount of 1 g to 15 g of 2′-FL and DFL; more preferably 2 g to 10 g. For example, the synthetic composition may contain 3 g to 7 g of 2′-FL and DFL.

The pack preferably comprises at least about 21 daily doses; for example, about 28 daily doses.

Each daily dose in the pack may also contain a bifidobacteria; for example, Bifidobacterium longum and/or Bifidobacterium bifidum.

A fourth aspect of this invention relates to a method for reducing pain and/or the perception of visceral pain in a non-infant human, the method comprising administering to the human an effective amount of a combination of the human milk oligosaccharides 2′-FL and DFL.

Preferably the severity of the pain or perception of pain is reduced; for example, by at least 40% as compared to the pain or perception of pain prior to administration of the 2′-FL and DFL. More preferably, the severity of the pain or perception of pain is reduced by at least 50% as compared to the pain or perception of pain prior to administration of the 2′-FL and DFL. Further, the number of days pain occurs or is perceived to occur is preferably reduced as compared to the number of days prior to administration of the 2′-FL and DFL. For example, the number of days pain occurs or is perceived to occur over the last 10 days may decrease by at least 40%, more preferably by at least 50%. For example, the number of days of pain over the last 10 days may decrease from greater than 5 days to less than 3 days.

In one embodiment, the human suffers from a functional gastrointestinal disorder; for example, irritable bowel syndrome (MS patient), abdominal migraine, or a non-coeliac wheat sensitivity. In another embodiment, the human suffers from a gastrointestinal disease, for example, Crohn's Disease, ulcerative colitis, and/or infectious diarrhea.

Preferably the amount of 2′-FL and DFL is effective to (i) increase the abundance, particularly the relative abundance, of bifidobacteria, and/or (ii) improve the gut barrier function of the human. More preferably the 2′-FL and DFL are administered to the non-infant human:

(a) in a first step for a period of 14 days or less for increasing the relative abundance of bifidobacteria of the phylogenetic Bifidobacterium adolescentis group; and

(b) in a second step for an additional period for increasing the relative abundance of Bifidobacterium longum and/or Bifidobacterium bifidum,

in the microbiota in the gastrointestinal tract of the non-infant human. The patient may have intestinal dysbiosis and/or an impaired mucosal barrier.

Preferably, the 2′-FL and DFL are present in a mass ratio of about 95:5 to about 75:25, preferably about 90:10 to about 80:20. For example, the 2′-FL and DFL are present in a mass ratio of about 89:19 to about 83:17.

The synthetic composition can be a nutritional or pharmaceutical composition. Preferably, the synthetic composition is administered daily. Furthermore, the synthetic composition is preferably administered for a period of at least one month, such as at least 2 months or for a longer period of time, for example chronically on an ongoing basis.

The synthetic composition may be administered to the human as a daily dose of about 1 g to about 15 g such as from about 3 g to about 10 g of 2′-FL and DFL. The patient can be administered a higher amount, preferably 5 g to 10 g per day, of the 2′-FL and DFL for an initial treatment period, followed by a lower amount, preferably 3 g to 5 g per day, for a maintenance period. The initial treatment period can be 1 to 8 weeks. The maintenance period is at least 1 month.

### BRIEF DESCRIPTION OF THE FIGURES

A more particular description of the embodiments briefly described above will be rendered by reference to specific embodiments that are illustrated in the appended drawings. Understanding that these drawings depict only some embodiments and are not, therefore, to be considered to be limiting of scope, the embodiments will be described and explained with additional specificity and detail through the use of the accompanying drawings, in which:

FIG. 1 illustrates one example embodiment where the amplitude of contraction measured as a function of colorectal dilatation in non-sensitized mice (AIN93; dots), in sensitized mice (was:AIN93; squares) and in sensitized mice to which 2′-FL and DFL have been administered (was:2′-FL+DFL; diamonds);

FIG. 2 illustrates one example embodiment where the amplitude of contraction measured as a function of colorectal dilatation in non-sensitized mice (AIN93; dots), in sensitized mice (was:AIN93; squares) and in sensitized mice to which 2′-FL, DFL, and LNT have been administered (was:2′-FL+DFL; diamonds);

FIG. 3 illustrates one example embodiment where amplitude of contraction measured as a function of colorectal dilatation in non-sensitized mice (AIN93; dots), in sensitized mice (was:AIN93; squares) and in sensitized mice to which 3′-SL and 6′-SL have been administered (was:3′-SL+6′-SL; diamonds); and

FIG. 4 illustrates one example embodiment where the amplitude of contraction measured as a function of colorectal dilatation in non-sensitized mice (AIN93; dots), in sensitized mice (was:AIN93; squares) and in sensitized mice to which 2′-FL, DFL, LNT, LNnT, 3′-SL and 6′-SL have been administered (was:6 HMOs; diamonds).

### DETAILED DESCRIPTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the various embodiments disclosed herein may be combined in any suitable manner in one or more embodiments. One skilled in the relevant art will recognize, however, that the various embodiments disclosed herein may be practiced without one or more of the specific details, or with other methods, components, materials, and so forth. In other instances, well-known structures, materials, or procedures are not shown or described in detail to avoid obscuring aspects of the various embodiments disclosed herein.

It has been surprisingly found that the combination of 2′-FL and DFL is to reduce visceral pain and/or the perception of visceral pain to a greater extent than other human milk oligosaccharides (HMOs) or combinations of HMOs. Further, the combination of 2′-FL and DFL preferentially increases the abundance of beneficial bacteria, such as bifidobacteria, and/or act to repair damage in the mucosal barrier. This may result in the regulation of immune responses and gut-brain interactions. Furthermore, 2′-FL and DFL may act on neuronally-dependent, gut motor complexes to address disorders of gut motility and possibly have beneficial effects on the central nervous systems. As an outcome, visceral hypersensitivity and/or pain is reduced.

In this specification, the following terms have the following meanings:

“Abdominal migraine” means a condition marked by episodic moderate to severe abdominal pain. The pain usually lasts from 1 hour to three days. There is typically complete normality between episodes. Abdominal migraine may have an early symptom (prodrome) indicating the onset of a disease or illness, e.g. constipation, mood changes food cravings, etc. The pain typically begins in the middle of the abdomen (belly) and is usually accompanied by symptoms such as little desire to eat, nausea, and vomiting. Other symptoms can include sensitivity to light, sensitivity to sound and dizziness. Abdominal migraine may be often followed by one or more symptoms that occur after the attack (postdrome) and last from a few hours to about 2-3 days, e.g. fatigue, mental confusion, skin and scalp sensitivity, mood change, etc. Abdominal migraine occurs mainly in children.

“Bifidobacterium of the B. adolescentis phylogenetic group” means a bacterium selected from a group consisting of Bifidobacterium adolescentis, Bifidobacterium angulatum, Bifidobacterium catenulatum, Bifidobacterium pseudocatenulatum, Bifidobacterium kashiwanohense, Bifidobacterium dentum and Bifidobacterium stercoris (Duranti et al. Appl. Environ. Microbiol. 79, 336 (2013), Bottacini et al. Microbial Cell Fact. 13:S4 (2014)). Preferably, a Bifidobacterium of the B. adolescentis phylogenetic group is Bifidobacterium adolescentis and/or Bifidobacterium pseudocatenulatum.

“Effective amount” means an amount of an HMO sufficient to render a desired outcome in a human. An effective amount can be administered in one or more doses to achieve the desired outcome.

“Enteral administration” means any conventional form for delivery of a composition to a patient that causes the deposition of the composition in the gastrointestinal tract (including the stomach). Methods of enteral administration include feeding through a nasogastric tube or jejunum tube, oral, sublingual and rectal.

“Human milk oligosaccharide” or “HMO” means a complex carbohydrate found in human breast milk (Urashima et al.: Milk Oligosaccharides. Nova Science Publisher (2011); Chen Adv. Carbohydr. Chem. Biochem. 72, 113 (2015)). The HMOs have a core structure comprising a lactose unit at the reducing end that can be elongated by one or more β-N-acetyl-lactosaminyl and/or one or β-more lacto-N-biosyl units, and which core structure can be substituted by an α L-fucopyranosyl and/or an α-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-fucopentaose Vi (LNFP-Vi) lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (FpLNnH II) and fucosyl-lacto-N-neohexaose (FLNnH). Examples of acidic HMOs include 3′-sialyllactose (3′-SL), 6′-sialyllactose (6′-SL), 3-fucosyl-3′-sialyllactose (FSL), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

“Irritable bowel syndrome” (abbreviated as IBS) means a functional bowel disorder in which recurrent abdominal pain is associated with defecation or a change in bowel habits. Disordered bowel habits are typically present (i.e., constipation, diarrhea or a mix of constipation and diarrhea), as are symptoms of abdominal bloating/distension. When diagnosed under Rome IV criteria, the patient must experience recurrent abdominal pain on average at least 1 day/week in the last 3 months, and the pain must be associated with two or more of the following criteria: (1) related to defecation; (2) associated with a change in the frequency of stool; and (3) associated with a change in the form (appearance) of stool. Symptom onset should occur at least 6 months prior to diagnosis and symptoms should be present during the last 3 months.

“Microbiota”, “microflora” and “microbiome” mean a community of living microorganisms that typically inhabit a bodily organ or part, particularly the gastrointestinal organs of humans. The most dominant members of the gastrointestinal microbiota include microorganisms of the phyla of Firmicutes, Bacteroidetes, Actinobacteria, Proteobacteria, Synergistetes, Verrucomicrobia, Fusobacteria, and Euryarchaeota; at genus level Bacteroides, Faecalibacterium, Bifidobacterium, Roseburia, Ali stipes, Collinsella, Blautia, Coprococcus, Ruminococcus, Eubacterium and Dorea; at species level Bacteroides uniformis, Alistipes putredinis, Parabacteroides merdae, Ruminococcus bromii, Dorea longicatena, Bacteroides caccae, Bacteroides thetaiotaomicron, Eubacterium hallii, Ruminococcus torques, Faecalibacterium prausnitzii, Ruminococcus lactaris, Collinsella aerofaciens, Dorea formicigenerans, Bacteroides vulgatus and Roseburia intestinalis. The gastrointestinal microbiota includes the mucosa-associated microbiota, which is located in or attached to the mucous layer covering the epithelium of the gastrointestinal tract, and luminal-associated microbiota, which is found in the lumen of the gastrointestinal tract.

“Modulating of microbiota” means exerting a modifying or controlling influence on microbiota, for example, an influence leading to an increase in the indigenous intestinal abundance of Bifidobacterium, Barnesiella, Faecalibacterium and/or other butyrate-producing bacteria. In another example, the influence may lead to a reduction of the intestinal abundance of Ruminococcus gnavus and/or Proteobacteria. “Proteobacteria” are a phylum of Gram-negative bacteria and include a wide variety of pathogenic bacteria, such as Escherichia, Salmonella, Vibrio, Helicobacter, Yersinia, and many other notable genera.

“Non-infant human” or “non-infant” means a human of 3 years of age and older. A non-infant human can be a child, a teenager, an adult or an elderly person.

“Non-coeliac wheat sensitivity” means is a syndrome characterized by intestinal and extra-intestinal symptoms related to the ingestion of gluten-containing food, in subjects that are not affected by either coeliac disease or wheat allergy. “Non-coeliac gluten sensitivity” has the same meaning and the two terms are used interchangeably. The gluten-containing food usually contains a gluten-containing cereal such as wheat, barley, and rye.

“Oral administration” means any conventional form for the delivery of a composition to a human through the mouth. Accordingly, oral administration is a form of enteral administration.

“Preventive treatment” or “prevention” in the present context means treatment is given or action taken to diminish the risk of onset or recurrence of a disease.

“Prophylactically reducing symptom severity and/or occurrence” means reducing the severity and/or occurrence of symptoms at a later point in time.

“Relative abundance of a bifidobacteria” means the abundance of a bifidobacteria species relative to other bifidobacteria in the microbiota of the gastrointestinal tract of humans.

“Relative growth of a bifidobacteria” means the growth of a Bifidobacterium species relative to other bifidobacteria in the microbiota in the gastrointestinal tract of humans.

“Secondary prevention” means prevention of the onset of the condition in a high-risk patient, or prevention of the reoccurrence of symptoms in a patient who has already has the condition. A “high-risk” patient is an individual who is predisposed to developing the condition; for example, a person with a family history of the condition.

“Synthetic composition” means a composition that is artificially prepared and preferably means a composition containing at least one compound that is produced ex vivo chemically and/or biologically, e.g. by means of chemical reaction, enzymatic reaction or recombinantly. In some embodiments, a synthetic composition of the various embodiments disclosed herein may be, but preferably is not, identical with a naturally occurring composition. The synthetic composition typically comprises one or more compounds, including one or more HMOs, that are capable of reducing IBS symptoms in a lactose intolerant IBS patient. Also, in some embodiments, the synthetic compositions may comprise one or more nutritionally or pharmaceutically active components which do not affect adversely the efficacy of the above-mentioned compounds. Some non-limiting embodiments of a synthetic composition of the various embodiments disclosed herein are also described below.

“Treat” means to address a medical condition or disease with the objective of improving or stabilizing an outcome in the person being treated or addressing an underlying nutritional need. Treat therefore includes the dietary or nutritional management of the medical condition or disease by addressing the nutritional needs of the person being treated. “Treating” and “treatment” have grammatically corresponding meanings.

“Therapy” means treatment is given or action is taken to reduce or eliminate symptoms of a disease or pathological condition.

“Visceral hypersensitivity” means an increased intensity of sensation of stimuli of visceral organs of the body. In particular, a person who experiences visceral hypersensitivity may have a lowered threshold for visceral pain, such as abdominal pain and discomfort in response to pressure, stimulation or distension within the abdomen.

“Visceral organ” is an organ of the digestive, respiratory, urogenital and endocrine systems as well as the spleen, the heart, and great vessels.

“Visceral pain” means a pain or the perception of pain that results from the activation of nociceptors in a visceral organ. The pain is an unpleasant sensation that can range from mild discomfort to agony.

The combination of 2′-FL and DFL used in the synthetic composition, pack, and method of the various embodiments disclosed herein can be isolated or enriched by well-known processes from milk(s) secreted by mammals including, but not limited to human, bovine, ovine, porcine, or caprine species. HMOs can also be produced by well-known processes using microbial fermentation, enzymatic processes, chemical synthesis, or combinations of these technologies. As examples, 2′-FL can be made chemically as described in WO 2010/115934 and WO 2010/115935. As examples of enzymatic production, sialylated oligosaccharides can be made as described in WO 2012/007588, fucosylated oligosaccharides can be made as described in WO 2012/127410, and advantageously diversified blends of human milk oligosaccharides can be made as described in WO 2012/156897 and WO 2012/156898. Biotechnological methods that describe how to make core (non-fucosylated neutral) human milk oligosaccharides optionally substituted by fucose or sialic acid using genetically modified E. coli con be found in WO 01/04341 and WO 2007/101862. In particular, WO2015/032413, WO2015/032412, and WO2016/095924 describe biotechnological processes for the manufacture of 2′-FL and DFL.

The combination of 2′-FL and DFL in any of the aspects of the present invention is a mixture consisting essentially of or consisting of 2′-FL and DFL. In one embodiment, the combination/mixture of 2′-FL and DFL is used as it is (neat), without any carrier and/or diluent. In other embodiment, the combination/mixture of 2′-FL and DFL is used in a synthetic composition with one or more inert carriers/diluents that are acceptable in nutritional or pharmaceutical compositions, for example solvents (e.g. water, water/ethanol, oil, water/oil), dispersants, coatings, absorption promoting agents, controlled release agents, inert excipients (e.g. starches, polyols, granulating agents, microcrystalline cellulose, diluents, lubricants, binders, and disintegrating agents). These compositions do not contain HMOs other than 2′-FL and DFL. In other embodiments, the combination/mixture of 2′-FL and DFL is used in a synthetic pharmaceutical or nutritional composition, as disclosed below. The synthetic pharmaceutical or nutritional composition does not contain HMOs other than 2′-FL and DFL.

The synthetic composition can take any suitable form. For example, the synthetic composition can be in the form of a nutritional composition such as a food composition, a rehydration solution, a medical food or food for special medical purposes, a nutritional supplement and the like. The nutritional composition can contain sources of protein, lipids and/or digestible carbohydrates and can be in powdered or liquid forms. The composition can be designed to be the sole source of nutrition or as a nutritional supplement in unit dose form. Other forms of the synthetic composition include a pharmaceutical composition in unit dose form.

Suitable protein sources include milk proteins, soy protein, rice protein, pea protein, and oat protein, or mixtures thereof. Milk proteins can be in the form of milk protein concentrates, milk protein isolates, whey protein or casein, or mixtures of both. The protein can be a whole protein or hydrolyzed protein, either partially hydrolyzed or extensively hydrolyzed. Hydrolyzed protein offers the advantage of easier digestion which can be important for humans with inflamed or compromised GI tracts. The protein can also be provided in the form of free amino acids. The protein can comprise about 5% to about 30% of the energy of the nutritional composition, normally about 10% to 20%. Ideally, the source of protein does not include excessive amounts of lactose.

The protein source can be a source of glutamine, threonine, cysteine, serine, proline, or a combination of these amino acids. The glutamine source can be a glutamine dipeptide and/or a glutamine enriched protein. Glutamine can be included due to the use of glutamine by enterocytes as an energy source. Threonine, serine, and proline are important amino acids for the production of mucin. Mucin coats the GI tract and can improve intestinal barrier function and mucosal healing. Cysteine is a major precursor of glutathione, which is key for the antioxidant defenses of the body.

Suitable digestible carbohydrates include maltodextrin, hydrolysed or modified starch or corn starch, glucose polymers, corn syrup, corn syrup solids, high fructose corn syrup, rice-derived carbohydrates, pea-derived carbohydrates, potato-derived carbohydrates, tapioca, sucrose, glucose, fructose, sucrose, honey, sugar alcohols (e.g. maltitol, erythritol, sorbitol), or mixtures thereof. Preferably the composition is reduced in or free from added lactose or other FODMAP carbohydrates. Generally, digestible carbohydrates provide about 35% to about 55% of the energy of the nutritional composition. A suitable digestible carbohydrate is a low dextrose equivalent (DE) maltodextrin.

Suitable lipids include medium-chain triglycerides (MCT) and long-chain triglycerides (LCT). Preferably the lipid is a mixture of MCTs and LCTs. For example, MCTs can comprise about 30% to about 70% by weight of the lipids, more specifically about 50% to about 60% by weight. MCTs offer the advantage of easier digestion which can be important for humans with inflamed or compromised GI tracts. Generally, the lipids provide about 35% to about 50% of the energy of the nutritional composition. The lipids can contain essential fatty acids (omega-3 and omega-6 fatty acids). Preferably these polyunsaturated fatty acids provide less than about 30% of the total energy of the lipid source.

Suitable sources of long-chain triglycerides are rapeseed oil, sunflower seed oil, palm oil, soy oil, milk fat, corn oil, high oleic oils, and soy lecithin. Fractionated coconut oils are a suitable source of medium-chain triglycerides. The lipid profile of the nutritional composition is preferably designed to have a polyunsaturated fatty acid omega-6 (n-6) to omega-3 (n-3) ratio of about 4:1 to about 10:1. For example, the n-6 to n-3 fatty acid ratio can be about 6:1 to about 9:1.

The nutritional composition may also include vitamins and minerals. If the nutritional composition is intended to be a sole source of nutrition, it preferably includes a complete vitamin and mineral profile. Examples of vitamins include vitamins A, B-complex (such as B1, B2, B6, and B12), C, D, E and K, niacin and acid vitamins such as pantothenic acid, folic acid, and biotin. Examples of minerals include calcium, iron, zinc, magnesium, iodine, copper, phosphorus, manganese, potassium, chromium, molybdenum, selenium, nickel, tin, silicon, vanadium and boron.

The nutritional composition can also include a carotenoid such as lutein, lycopene, zeaxanthin, and beta-carotene. The total amount of carotenoid included can vary from about 0.001 μg/ml to about 10 μg/ml. Lutein can be included in an amount of from about 0.001 μg/ml to about 10 μg/ml, preferably from about 0.044 μg/ml to about 5 μg/ml of lutein. Lycopene can be included in an amount from about 0.001 μg/ml to about 10 μg/ml, preferably about 0.0185 μg/ml to about 5 μg/ml of lycopene. Beta-carotene can comprise from about 0.001 μg/ml to about 10 mg/ml, for example about 0.034 μg/ml to about 5 μg/ml of beta-carotene.

The nutritional composition preferably also contains reduced concentrations of sodium; for example, from about 300 mg/l to about 400 mg/l. The remaining electrolytes can be present in concentrations set to meet needs without providing an undue renal solute burden on kidney function. For example, potassium is preferably present in a range of about 1180 to about 1300 mg/1; and chloride is preferably present in a range of about 680 to about 800 mg/l.

The nutritional composition can also contain various other conventional ingredients such as preservatives, emulsifying agents, thickening agents, buffers, fibers, and prebiotics (e.g. fructooligosaccharides, galactooligosaccharides), probiotics (e.g. B. animalis subsp. lactis BB-12, B. lactis HNO19, B. lactis Bi07, B. infantis ATCC 15697, L. rhamnosus GG, L. rhamnosus HNOO1, L. acidophilus LA-5, L. acidophilus NCFM, L. fermentum CECT5716, B. longum BB536, B. longum AH1205, B. longum AH1206, B. breve M-16V, L. reuteri ATCC 55730, L. reuteri ATCC PTA-6485, L. reuteri DSM 17938), antioxidant/anti-inflammatory compounds including tocopherols, carotenoids, ascorbate/vitamin C, ascorbyl palmitate, polyphenols, glutathione, and superoxide dismutase (melon), other bioactive factors (e.g. growth hormones, cytokines, TFG-β), colorants, flavours, and stabilisers, lubricants, and so forth.

The nutritional composition can be formulated as a soluble powder, a liquid concentrate, or a ready-to-use formulation. The composition can be fed to a human in need via a nasogastric tube or orally. Various flavors and other additives can also be present.

The nutritional compositions can be prepared by any commonly used manufacturing techniques for preparing nutritional compositions in solid or liquid form. For example, the composition can be prepared by combining various feed solutions. A protein-in-fat feed solution can be prepared by heating and mixing the lipid source and then adding an emulsifier (e.g. lecithin), fat-soluble vitamins, and at least a portion of the protein source while heating and stirring. A carbohydrate feed solution is then prepared by adding minerals, trace, and ultra-trace minerals, thickening or suspending agents to water while heating and stirring. The resulting solution is held for 10 minutes with continued heat and agitation before adding carbohydrates (e.g. the 2′-FL and DFL and digestible carbohydrate sources). The resulting feed solutions are then blended together while heating and agitating and the pH adjusted to 6.6-7.0, after which the composition is subjected to high-temperature short-time processing during which the composition is heat-treated, emulsified and homogenized, and then allowed to cool. Water-soluble vitamins and ascorbic acid are added, the pH is adjusted to the desired range if necessary, flavors are added, and water is added to achieve the desired total solid level.

For a liquid product, the resulting solution can then be aseptically packed to form an aseptically packaged nutritional composition. In this form, the nutritional composition can be in ready-to-feed or concentrated liquid form. Alternatively, the composition can be spray-dried and processed and packaged as a reconstitutable powder.

When the nutritional product is a ready-to-feed nutritional liquid, it may be preferred that the total concentration of 2′-FL and DFL in the liquid, by weight of the liquid, is from about 0.1% to about 1.5%, including from about 0.2% to about 1.0%, for example from about 0.3% to about 0.7%. When the nutritional product is a concentrated nutritional liquid, it may be preferred that the total concentration of 2′-FL and DFL in the liquid, by weight of the liquid, is from about 0.2% to about 3.0%, including from about 0.4% to about 2.0%, for example from about 0.6% to about 1.5%.

In another embodiment, the nutritional composition is in a unit dosage form. The unit dosage form can contain an acceptable food-grade carrier, e.g. phosphate-buffered saline solution, mixtures of ethanol in water, water and emulsions such as an oil/water or water/oil emulsion, as well as various wetting agents or excipients. The unit dosage form can also contain other materials that do not produce an adverse, allergic or otherwise unwanted reaction when administered to a human. The carriers and other materials can include solvents, dispersants, coatings, absorption promoting agents, controlled release agents, and one or more inert excipients, such as starches, polyols, granulating agents, microcrystalline cellulose, diluents, lubricants, binders, and disintegrating agents. Preferably the unit dosage form comprises primarily 2′-FL and DFL with a minimum amount of binders and/or excipients. Unit dosage forms are particularly suitable when nutritionally incomplete or not intended as a sole source of nutrition.

A unit dosage form can be administered orally, e.g. as a tablet, capsule, or pellet containing a predetermined amount of the mixture, or as a powder or granules containing a predetermined concentration of the mixture or a gel, paste, solution, suspension, emulsion, syrup, bolus, electuary, or slurry, in an aqueous or non-aqueous liquid, containing a predetermined concentration of the mixture. An orally administered composition can include one or more binders, lubricants, inert diluents, flavoring agents, and humectants. An orally administered composition such as a tablet can optionally be coated and can be formulated to provide sustained, delayed or controlled release of the 2′-FL and DFL.

A unit dosage form can also be administered by nasogastric tube or direct infusion into the GI tract or stomach.

A unit dosage form can also include therapeutic agents such as antibiotics, probiotics, analgesics, and anti-inflammatory agents.

The proper dosage of a nutritional composition for a human can be determined in a conventional manner, based upon factors such as the concentration of 2′-FL and DFL, the human's condition, immune status, body weight, and age. In some cases, the dosage will be such that the 2′-FL and DFL is delivered at a concentration similar to that found in human breast milk. The required amount of 2′-FL and DFL would generally be in the range from about 1 g to about 15 g per day, in certain embodiments from about 2 g to about 10 g per day, for example about 3 g to about 7 g per day. Appropriate dose regimes can be determined by methods known to those skilled in the art.

In further embodiments, the 2′-FL and DFL can be formulated as a pharmaceutical composition. The pharmaceutical composition can contain a pharmaceutically acceptable carrier, e.g. phosphate-buffered saline solution, mixtures of ethanol in water, water and emulsions such as an oil/water or water/oil emulsion, as well as various wetting agents or excipients. The pharmaceutical composition can also contain other materials that do not produce an adverse, allergic or otherwise unwanted reaction when administered to a human. The carriers and other materials can include solvents, dispersants, coatings, absorption promoting agents, controlled release agents, and one or more inert excipients, such as starches, polyols, granulating agents, microcrystalline cellulose, diluents, lubricants, binders, and disintegrating agents.

The pharmaceutical compositions can be administered orally, e.g. as a tablet, capsule, or pellet containing a predetermined amount, or as a powder or granules containing a predetermined concentration or a gel, paste, solution, suspension, emulsion, syrup, bolus, electuary, or slurry, in an aqueous or non-aqueous liquid, containing a predetermined concentration. Orally administered compositions can include binders, lubricants, inert diluents, flavoring agents, and humectants. Orally administered compositions such as tablets can optionally be coated and can be formulated to provide sustained, delayed or controlled release of the mixture therein.

The pharmaceutical compositions can also be administered by rectal suppository, aerosol tube, nasogastric tube or direct infusion into the GI tract or stomach.

The pharmaceutical compositions can also include therapeutic agents such as antibiotics, probiotics, analgesics, and anti-inflammatory agents. The proper dosage of a pharmaceutical composition can be determined in a conventional manner, based upon factors such as the concentration of the 2′-FL and DFL, the patient's condition, immune status, body weight, and age. In some cases, the dosage will be such that the 2′-FL and DFL is delivered at a concentration similar to that found in human breast milk. The required amount of 2′-FL and DFL would generally be in the range from about 1 g to about 15 g per day, in certain embodiments from about 2 g to about 10 g per day, for example about 3 g to about 7 g per day. Appropriate dose regimes can be determined by methods known to those skilled in the art.

For reducing visceral pain or the perception of pain in a non-infant human, the amount of 2′-FL and DFL required to be administered will vary depending upon factors such as the risk and severity of the pain, any underlying medical condition or disease, age, the form of the composition, and other medications being administered. Further, the amount may vary depending upon whether the 2′-FL and DFL are being used prophylactically (when the dose may be higher) or whether the 2′-FL and DFL are being used during maintenance (when the dose may be lower). However, the required amount can be readily set by a medical practitioner and would generally be in the range from about 1 g to about 15 g per day, in certain embodiments from about 2 g to about 10 g per day, for example from about 3 g to about 7 g per day. An appropriate dose can be determined based on several factors, including, for example, body weight and/or condition, the severity of the pain being managed, other ailments and/or diseases, the incidence and/or severity of side effects and the manner of administration. Appropriate dose ranges may be determined by methods known to those skilled in the art. During an initial phase, the dosing can be higher (for example 3 g to 15 g per day, preferably 3 g to 10 g per day). During a maintenance phase, the dosing can be reduced (for example, 1 g to 10 g per day, preferably 2 g to 7.5 g per day).

The duration of the administration will vary depending upon factors such as the risk and severity of the medical condition, age, the form of the composition, the dose and other medications being administered. However, the duration can be readily set by a medical practitioner. Generally, a duration of at least a week will be required to sufficiently impact symptoms. For example, the duration may be for 1 to 3 months. The administration can continue chronically for an indefinite period.

### EXAMPLES

Examples are to illustrate various non-limiting embodiments disclosed herein.

### Example 1—Human Trial

A total of 60 male and female IBS patients are recruited to participate in the study. After a screening visit and run-in period of 1-2 weeks, the patients are selected. The patients are randomized into three groups, each of 20 patients, with two groups consuming the treatment product and one group the placebo product for 4 weeks. The treatment product contains either 5 or 10 grams of a combination of 2′-FL and DFL in an 85:15 mass ratio, while the placebo product contains 5 grams glucose. Both products are in powder form in a unit dosage container.

The patients are eligible to participate if they are at an age between 18-60 years, fulfill the definition of IBS-D, D3 S-C or D3 S-A/M according to the Rome IV criteria for IBS and have a global IBS-SSS score of >174 during the 2 weeks run-in period. All recruited patients are able and willing to understand and comply with the study procedures. Patients are excluded if: they have any known gastrointestinal disease(s) that may cause symptoms or interfere with the trial outcome, in particular, lactose intolerance and coeliac disease; they have participated in a clinical study one month prior to screening visit; they have abnormal results in the screening tests which are clinically relevant for study participation; they are suffering for a severe disease such as malignancy, diabetes, severe coronary disease, kidney disease, neurological disease, or severe psychiatric disease or any condition which can confound the results of the study; used highly dosed probiotic supplements (yoghurt allowed) for 1 months prior to the study; consumed antibiotic drugs 1 months prior to the study; consumed on a regular basis any medication that might interfere with symptom evaluation 2 weeks prior to the study; diagnosed with and treated for IBS for more than 10 years; and pregnant or lactating.

At the screening visit, clinical and medical history and concomitant medication are registered. IBS diagnostic criteria will be assessed and part 2 of the IBS-SSS questionnaire will be completed.

A fecal sample kit is distributed together with the Bristol Stool Form Scale (B SFS) and Bowel Movement Diary (BMD) to be filled in during the 7 days just prior to visit 2. Patients will be asked to register their diet 3 days just prior to visit 2, and will be reminded not to change their usual diet during the study.

At the second visit, eligibility criteria are checked and eligible subjects are randomized to the three arms in the trial. A physical examination is done and a number of questionnaires (GSRS-IBS, IBS-SSS, HADS, NRS-11, VSI, IBS-QOL, and PHQ-15 scales) are answered. Questionnaires are filled in electronically. Those who are unable or unwilling to use the electronic system fill out the questionnaires on paper. Based on clinical symptoms and data from questionnaires, patients are characterized into one of the three following groups; diarrhea-predominant (IBS-D), constipation-predominant (IBS-C) or alternating/mixed (IBS-A/M). This enables the allocation of patients from each subgroup into the intervention groups. When allocated to the groups, the patients are provided with either treatment or placebo products. Sigmoidoscopy is performed and mucosal biopsies and fecal aspirates taken. Patients are asked about any adverse events and any changes in their usual medication. The BSFS and BMD are collected and new forms, to be filled in daily during the intervention period, are distributed. Fecal samples are collected and equipment for new samples is distributed. Blood samples are collected for routine clinical chemistry and hematology and biomarker analysis and a saliva sample is collected to analyze FUT2 secretor status. Diet records are collected, and patients are asked to register their diet for 3 days just prior to visit 3. Patients are reminded not to change their usual diet during the study.

At the third visit, a physical examination is performed and a number of questionnaires (GSRS-IBS, IBS-SSS, HADS, NRS-11, VSI, IBS-QOL, and PHQ-15 scales) are answered. Questionnaires are filled in electronically. Those who are unable or unwilling to use the electronic system fill out the questionnaires on paper. Remaining study products and compliance diaries are collected to check compliance. Blood samples are collected for routine clinical chemistry and hematology and biomarker analysis, and sigmoidoscopy is performed and mucosal biopsies and fecal aspirates taken. Patients are asked about any adverse events and any changes in their usual medication. Fecal samples are collected and equipment for collecting new samples distributed. The BSFS and BMD are collected and new forms, to be filled in during the 7 days just prior to visit 4, are distributed. Diet records are collected, and patients are reminded not to change their usual diet during the study.

The study runs for 8 weeks with the patients consuming either a placebo or a treatment product daily for 4 weeks. Patients are instructed to consume the products in the morning with breakfast. Compliance is monitored through the interactive internet-enabled system.

At the end of the study, each patient has an exit visit with the medical team. Fecal samples and blood samples are collected and analyzed as before. A number of questionnaires (GSRS-IBS, IBS-SSS, HADS, NRS-11, VSI, IBS-QOL, and PHQ-15 scales) are answered. Questionnaires are filled in electronically. Those who are unable or unwilling to use the electronic system fill out the questionnaires on paper. Patients are asked about any adverse events and any changes in their usual medication or diet, and The BSFS and BMD are collected.

To assess the microbiota profile, DNA is extracted from fecal samples using a 96-well PowerSoil DNA Isolation Kit (MO-BIO). A minimum of one sample-well per plate is kept empty to serve as a negative control during PCR. PCR is done with the forward primer S-D-Bact-0341-b-S-17 and reverse primer S-D-Bact-0785-a-A-21 with Illumina adapters attached (Klindworth et al. Nucleic Acids Res. 41, el (2013)). These are universal bacterial 16S rDNA primers, which target the V3-V4 region. Following PCR program is used: 98° C. for 30 sec, 25× (98° C. for 10 s, 55° C. for 20 s, 72° C. for 20 s), 72° C. for 5 min. Amplification is verified by running the products on a 1% agarose gel. Barcodes are added in a nested PCR using the Nextera Index Kit V2 (Illumina) with the following PCR program: 98° C. for 30 sec, 8× (98° C. for 10 s, 55° C. for 20 s, 72° C. for 20 s), 72° C. for 5 min. Attachment of primers is verified by running the products on a 1% agarose gel. Products from the nested PCR are normalized using the SequalPrep Normalization Plate Kit and pooled. Pooled libraries are concentrated by evaporation and the DNA concentration of pooled libraries is measured on a Qubit fluorometer using the Qubit High Sensitivity Assay Kit (Thermo Fisher Scientific). Sequencing is done on a MiSeq desktop sequencer using the MiSeq Reagent Kit V3 (Illumina) for 2×300 bp paired-end sequencing. The 64-bit version of USEARCH is used for the bioinformatical analysis of the sequence data.

The results show that oral ingestion of 2′-FL and DFL modulate the intestinal microbiota, and specifically stimulate the abundance of bifidobacteria. The blood biomarker analysis indicates that the treatment patients have reduced levels of inflammatory markers, and the biopsy analysis reveals a reduction in activated mast cell meaning stabilization of mast cells. Reduction in visceral pain and an improvement in bowel movement are reported by treatment patients as compared to the placebo group. Collectively, 2′-FL and DFL are able to increase bifidobacteria and stabilize mast cells, and hereby contribute to improvement in visceral pain in IBS patients.

### Example 2—Mouse Model

The effectiveness of a combination of 2′-FL and DFL in reducing nociception is assessed in vivo in mice and compared to other mixes of human milk oligosaccharides.

C57Bl.6/J mice are purchased from Janvier SA (Le Genest St. Isle, France). Upon arrival, they are kept in polypropylene cages in a temperature-controlled room with a 12-hour dark-light cycle for 1 week. To measure abdominal muscle contractions as an index of pain, mice are equipped with 2 nickel-chrome wire electrodes, one implanted into the abdominal external oblique muscle and one under the skin of the abdomen. Surgery is performed under xylazine/ketamine anesthesia (both 1.2 mg, subcutaneously).

Two days after surgery, the mice are separated into 6 groups and each group is fed a different diet as follows:

Group 1: Control diet (AIN93);

Group 2: Control diet (AIN93);

Group 3: Experimental diet (2′-FL+DFL), AIN93 supplemented with 2′-FL 0.55 w/w % and DFL 0.06 w/w %;

Group 4: Experimental diet (2′-FL+DFL+LNT), AIN93 supplemented with 2′-FL 0.55 w/w %, DFL 0.06 w/w % and LNT 0.18 w/w %;

Group 5: Experimental diet (3′-SL+6′-SL), AIN93 supplemented with 3′-SL 0.07 w/w % and 6′-SL 0.09 w/w %;

Group 6: Experimental diet (6 HMOs), AIN93 supplemented with 2′-FL 0.55 w/w %, DFL 0.06 w/w %, 3′-SL 0.07 w/w %, 6′-SL 0.09% w/w, LNnT 0.05 w/w % and LNT 0.18 w/w %.

Five days after surgery, the mice in groups 2 to 6 are submitted to daily water avoidance stress (was) by placing them individually for 1 hour per day on a 3×3 cm platform in a 40×40 cm size pool filled with cold tap water up to 1 cm from the top of the platform. The mice in group 1 are subjected to a sham water avoidance stress by placing them for 1 hour per day on the platform in a waterless pool. The water avoidance stress and sham water avoidance stress is continued for 9 days. Animals have free access to water and food during this time.

A small balloon (Fogarty, Edwards Laboratories Inc., Santa Anna, USA) is introduced in the rectum of each mouse and fixed at the base of the tail. The balloon is progressively inflated from 0 to 0.10 ml, by steps of 0.02 ml. Each step of distension lasts 10 sec alternated with a 5 min recovery period in between without distension. Visceral sensitivity to colorectal distension (CRD) is assessed by abdominal muscle electromyography. The amplitude of contraction detected by electromyography is associated with pain perceived by the mice.

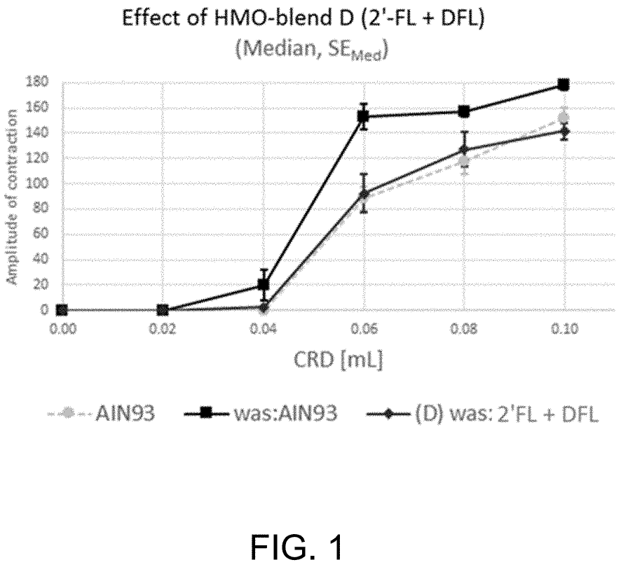

The amplitude of contraction is significantly increased by the water avoidance stress when comparing the results of the mice in Group 1 compared to the mice in Group 2. As shown in FIG. 1, the administration of a blend of 2′-FL and DFL is able to reduce the amplitude of contraction and thus the perceived pain of the mice (Group 3). For a CRD of 0.06 mL and above, the amplitude of contraction is not statistically distinguishable from what is observed in Group 1 mice. A CRD of 0.06 ml corresponds to the typical threshold of pain, whereas smaller dilatation volumes are associated with discomfort.

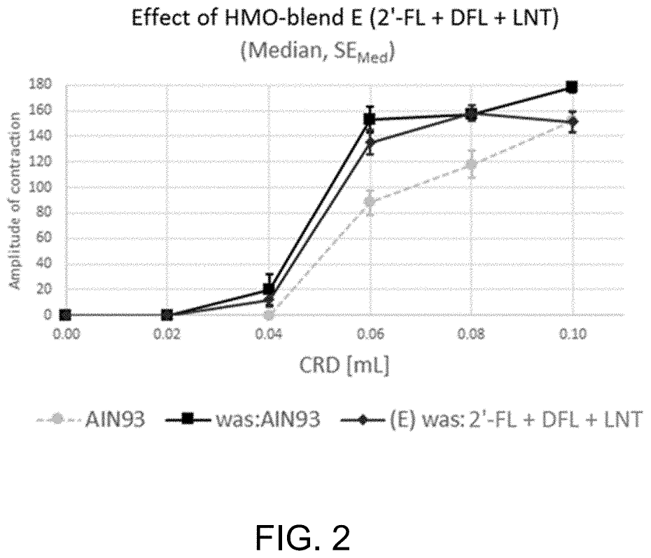

In contrast, the amplitude of contraction is reduced to a lesser extent for the other HMO mixes. In particular, the addition of LNT to 2′-FL and DFL (Group 4) reduced the reduction of the amplitude of contraction. The amplitude of contraction of Group 4 cannot be statistically distinguished from the Group 2 mice which did not receive 2′-FL and DFL (FIG. 2). Thus, the positive effect of 2′-FL and DFL on pain perception is lost when LNT is added, as can be seen by comparing FIGS. 1 and 2.

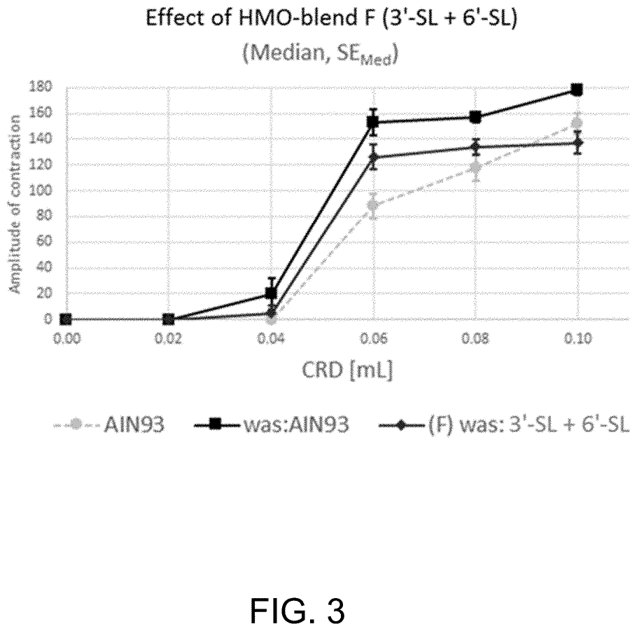

Administration of sialylated oligosaccharides 3′-SL and 6′-SL (Group 5) also results in a much smaller reduction of the amplitude of contraction and thus of the perception of pain compared to the mix consisting of 2′-FL and DFL (FIG. 3).

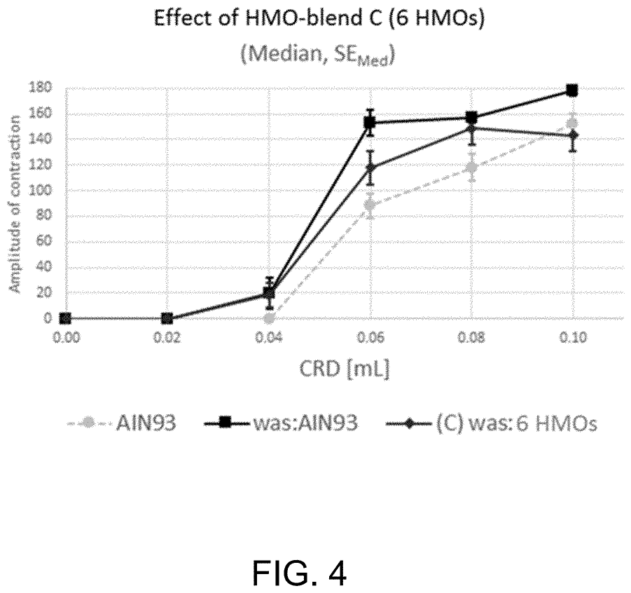

Similarly the mix of six HMOs 2′-FL, DFL, 3′-SL, 6′-SL, LNT and LNnT (Group 6) also reduces the amplitude of contraction and thus the perception of pain to a lesser extent than the mix of 2′-FL and DFL alone, in particular at a CRD of 0.06 or above (FIG. 4).

### Example 3—Nutritional Composition

A ready to feed nutritional composition is prepared from water, maltodextrin, corn syrup, sugar, milk protein concentrate, vegetable oil (canola, high oleic sunflower and corn), soy protein isolate, acacia gum, flavours, 2′-FL and DFL, potassium citrate, magnesium phosphate, cellulose gel and gum, calcium carbonate, sodium ascorbate, soy lecithin, choline bitartrate, calcium phosphate, alpha-tocopheryl acetate, ascorbic acid, carrageenan gum, ferric pyrophosphate, flavours, sweeteners (Stevia), vitamin A palmitate, niacinamide, vitamin D3, calcium pantothenate, manganese sulphate, copper sulphate, pyridoxine hydrochloride, thiamine hydrochloride, beta carotene, riboflavin, chromium chloride, folic acid, biotin, potassium iodide, phytonadione, sodium selenite, sodium molybdate, vitamin B12.

The composition provides a nutritional supplement that is a good source of protein, low in fat, vitamins, minerals, and antioxidants, and meets FODMAP criteria. Further, the composition contains 2′-FL and DFL which are able to promote the growth of beneficial intestinal bacteria and improve gut barrier function.

### Example 4—Capsule Composition

A capsule is prepared by filling about 1 g of 2′-FL and DFL into a 000 gelatine capsule using a filing machine. The capsules are then closed. The 2′-FL and DFL is in free-flowing, powder form.

### Example 5—Mucosal Barrier Function

The mix of 2′-FL and DFL is tested with respect to their ability to induce MUC2, TFF3, EIMβ, CHST5, and GAL3ST2 expression in the human LS174T cell culture model of goblet cells. The human LS174T cell line is obtained from the American Type Culture Collection (ATCC). LS174T cells are maintained in minimum essential medium (MEM) supplemented according to instructions at 37° C. in 5% CO2. 2′-FL and DFL are dissolved in cell culture grade water to the required concentration. The LS174T cells are treated with the 2′-FL and DFL solution containing 0 or 5 mg HMO/ml.

The LS174T cells are collected and suspended in Trizol reagent and total RNA is isolated using an RNA analysis kit (Qiagen) according to the manufacturer's instructions and the RNA isolates are quantified using Nanodrop analysis (Thermo Fisher Scientific). RNA isolates are reverse transcribed using a high capacity cDNA Reverse Transcription Kit (Applied Biosystems) to create cDNA, which is then used to assess gene expression via quantitative RT-PCR.

For the quantitative RT-PCR, specific TaqMan™ gene expression assays are obtained from Applied Biosystems, which include expression assays for MUC2, TFF3, CHST5, and GAL3 ST2. Quantitative real-time PCR is performed using TaqMan™ PCR Master Mix (Applied Biosystems). Reactions are run in duplicates in a 384-well plate using an Applied Biosystems 7900HT Fast Real-Time PCR System. The results are analyzed using SDS 2.3 software and calculated by the delta Ct method. All samples are normalized to Gus-β expression and fold induction is calculated over untreated controls. Gene expression is expressed as a fold increase compared to HMO-free control cells. The experiment is repeated three times.

The results indicate that treatment with 2′-FL and DFL increases the expression of the MUC2 and TFF3 genes compared to control cultures. Increased expression of goblet cell genes is specific and not universal, as evidenced by the minimal induction or lack of induction of CHST5 and GAL3 ST2, respectively. MUC2 and TFF3 are key components of the mucosal barrier and improve the mucosal barrier function.

What is claimed is:

1. A method comprising:
administering to a non-infant human an effective amount of a combination of the human milk oligosaccharides (“HMO”s) 2′-FL and DFL, wherein the amount of the combination of HMOs is effective for:
modulating the microbiota of the non-infant human; and
reducing one or more post-treatment condition selected from pain and a perception of visceral pain in the non-infant human.

2. The method of claim 1, wherein a severity of the selected post-treatment condition is reduced by at least 40% as compared to a corresponding pre-treatment condition prior to administration of the 2′-FL and DFL.

3. The method of claim 1, wherein a post-treatment period in which the non-infant human experiences the post-treatment condition is reduced as compared to a pre-treatment period of the pre-treatment condition prior to the administration of the HMOs 2′-FL and DFL.

4. The method of claim 3, wherein the last 10 days of the post-treatment period in which the non-infant human experiences the post-treatment condition decreases by at least 40%, and more preferably decreases by at least 50%.

5. The method of claim 1, wherein the non-infant human suffers from a functional gastrointestinal disorder selected from irritable bowel syndrome (MS patient), abdominal migraine, non-coeliac wheat sensitivity, and combinations thereof.

6. The method of claim 1, wherein the non-infant human suffers a gastrointestinal disease.

7. The method of claim 6, wherein the gastrointestinal disease is selected from Crohn's Disease, ulcerative colitis, infectious diarrhea, and combinations thereof.
